(19) United States
(12) Patent Application Publication
(10) Pub. No.: US 2020/0138495 A1
(43) Pub. Date: May 7, 2020

(54) IMPLANT FIXATION

(71) Applicant: SpineWelding AG, Schlieren (CH)

(72) Inventors: Jörg MAYER, Niederlenz (CH); Johan VAN HAVERMAET, Deinze (BE)

(21) Appl. No.: 16/483,562

(22) PCT Filed: Feb. 5, 2018
(86) PCT No.: PCT/EP2018/052827

§ 371 Date: Aug. 5, 2019

(30) Foreign Application Priority Data
Feb. 7, 2017 (CH) 00140/17

(51) Int. Cl. A61B 17/86; A61B 17/70; A61B 17/80

(52) U.S. Cl. CPC A61B 17/866; A61B 17/7059; A61B 2017/00955; A61B 17/7032; A61B 17/864; A61B 17/8047

(57) ABSTRACT

A spinal implant system with a spinal anchor implant, for example a pedicle screw, that has a head portion forming the proximal end. Further, the system includes a support plate, which is shaped to be placed against the posterior or lateral side of the vertebra and having an anchor receiving structure. The anchor implant extends through the anchor receiving structure into the bone tissue when the anchor implant is anchored in the bone tissue with the support plate between the head portion and the bone tissue. The system also has a thermoplastic material in a solid state, which is shaped and equipped to be placed relative to the anchored anchor implant and the support plate and to be made flowable by the impact of impinging energy so as to bridge and at least partially fill a gap between the anchor implant and the support plate.

### BACKGROUND OF THE INVENTION

### Field of the Invention

The invention is in the field of spinal implant systems.

### Description of Related Art

Spinal implant systems include systems in which different vertebrae are stabilized with respect to each other by a plate or rod. The anchors in the bone tissue to which the plate or rod is attached, is often a bone screw. In posterior or lateral systems that include attaching the implant to a posterior or lateral side of the spinal column, such screws are often pedicle screws that are introduced from the posterior side and reach through the pedicle into the vertebral body.

Especially for patients with comparably weak bone tissue and/or patients with a strong deformation of the spinal column to be corrected by the stabilization system, there is a risk that the screw loosens during deformity correction or in early loading proximally in the pedicle and/or distally in the vertebral body.

In accordance with the prior art, washers are suggested for distributing the relative forces acting between the screw and the bone tissue. However, washers supported by the plate/rod itself do not address the issue of mechanical overload on portions of the bone tissue, and washers supported by the bone tissue have only a small effect and in many situations do not work for monoaxial screw heads because these need to be oriented relative to the plate/rod and cannot be pressed against the washer. Monoaxial screw heads are especially preferred to multiaxial screw heads in situations in which high correction forces are necessary. Further, washers only have an effect if a certain pre-tension can be applied. However, there are situations in which such pre-tension relaxes or cannot be applied (or applied to an insufficient extent only) due to viscoelastic relaxation of the bone tissue or also because of a small bone density.

US 2016/0135961 discloses implants and endoprostheses suitable to be implanted in tissue and including two parts to be joined in situ. In a group of embodiments, the parts are a bearing and a movable part that are locked relative to each other especially with respect to relative rotation. This is done by a thermoplastic locking part introduced into an opening of the bearing and by applying mechanical vibration until the locking part is liquefied and pressed between the bearing and the movable part and into structures of the parts to yield a positive fit connection. This approach, however, requires a bearing to be composed of two bearing parts (half shells) to be closed around the movable part. It is not suited for replacement of prior art washers. Also, there is only minimal fixation with respect to axial movements, the thinner the half shells the less.

WO 2009/117837 discloses surgical devices for osteosynthesis. The devices include a bone plate and a load-bearing pin-like fastener with a head, the outer surface of which is coated by a bonding agent that is liquefiable by heat. An inner surface of a through hole in the plate, through which the fastener is inserted, and/or an outer surface of the head is provided with a structuring for enabling a positive fit between the fastener and the fixation element. Instead of being provided as a coating of the head, the bonding agent may also be provided as a coating of the inner surface of the through hole in the bone plate, or it may be externally provided during the fixation process or it may be provided in another region of the fastener and be transferred to the bonding region during fixation.

US 2010/0114097 shows a device for treating bone including a rigid body (for example, bone fixation plate) including a polymeric material extending over at least a target portion thereof, and further including a pin-like so-called locking element. The locking element includes a polymeric coating. After placing the locking element relative to the rigid body, the locking element is supplied with energy until a portion of the polymeric material of the rigid body is bonded (welded) to the coating of the locking element. This approach requires a physical contact between the locking element and the rigid body for the bonding (welding) process to take place.

### SUMMARY OF THE INVENTION

It would therefore be desirable to have a spinal implant system to be secured to the posterior or lateral side of a human spine, which system overcomes drawbacks of prior art spinal implant systems and which is especially suited for stabilization of a spinal column if high corrective forces have to be applied and/or if the bone tissue is comparably weak. It is also an object of the invention to provide a method for manufacturing such system. It is a further object of the present invention to provide a surgical method overcoming disadvantages of the prior art.

The spinal implant system according to the invention includes:
A spinal anchor implant, extending between a proximal end and a distal end and shaped to be anchored in a human spine with the distal end extending into the bone tissue of a human vertebra from a posterior or lateral side thereof, the spinal anchor implant having a proximal portion forming the proximal end,
And a support plate, the support plate being shaped to be placed against the posterior or lateral side of the vertebra, the support plate having an anchor receiving structure, wherein the anchor implant extends through the anchor receiving structure into the bone tissue when the anchor implant is anchored in the bone tissue,
wherein the implant system includes a thermoplastic material in a solid state, the thermoplastic material being shaped and equipped to be placed relative to the anchored anchor implant and the support plate and to be made flowable by the impact of impinging energy so as to bridge and at least partially fill a gap between the anchor implant and the support plate,
whereby after re-solidification of the thermoplastic material, the support plate supports the anchored anchor implant in a force transmitting manner via the thermoplastic material.

In this, the support plate is a plate in the sense that it often cannot be too thick but must be plate-like, with an extended surface portion bearing on the surface of the bone tissue. Also, the property of being a plate may imply that the support plate is one-piece (with the possible exception of a thermoplastic portion including the thermoplastic material) and, for example, contiguously extends around the anchor receiving structure if the same includes a through opening.

By having a flattish portion shaped to bear on the bone tissue, and by the shape of the anchor receiving location, the support plate may be suitable to take up bending forces on the anchor implant.

The proximal portion especially may be a head portion. Then, the support plate is shaped to be placed against the posterior or lateral side of the vertebra, wherein the anchor implant extends through the anchor receiving structure into the bone tissue when the anchor implant is anchored in the bone tissue with the support plate between the head portion and the bone tissue.

Alternatively, the proximal portion may include another connecting system for the support plate, for example an external thread cooperating with a fastening element. Especially (but not only) in embodiments without a head portion, it may be advantageous if the anchor implant includes a structure for axial stabilization relative to the support plate. Such structure for axial stabilization may include at least one of:
an undercut structure for being penetrated by the thermoplastic material, whereby after re-solidifications the portions of the thermoplastic material that have interpenetrated the undercut structure secure the anchor implant by a positive fit connection; such undercut including an undercut with respect to axial directions;
a structure for axial stabilization with respect to the bone tissue. Such structure may include an external thread in the enossal region (in the—distal—region of the anchor implant that reaches into the bone tissue), or a helical twist (as for example disclosed in WO 2011/054124, and/or thermoplastic material that interpenetrates the bone tissue thereby anchoring, after re-solidification, the anchor implant in the bone tissue.

The anchor implant may especially be configured for depth-effective anchoring and thus be elongate extending between the proximal end and the distal end. In an example, the anchor implant is a screw, especially a pedicle screw.

In accordance with an option, the anchor implant may be an anchor implant as disclosed in WO 2011/054124, especially as defined in any one of claims 1-12 thereof and as shown in the figures, namely a pedicle anchor device that has a longitudinal bore extending distally from a proximal end, and at least one hole from the longitudinal bore outward, wherein liquefiable thermoplastic material may be pressed through the longitudinal bore and through the hole into cancellous bone tissue to have, after re-solidification, an anchoring effect.

The thermoplastic material may be present as initially separate thermoplastic element or in the form of a plurality of initially separate elements. For example, the thermoplastic element may be pin-shaped or have another generic shape. Alternatively, the thermoplastic element/elements may have a shape adapted to the shape of the anchor implant and/or the support plate and thereby to the gap.

In embodiments that include thermoplastic material in form of at least one initially separate thermoplastic element, the support plate may include a guiding structure (guiding through hole/guiding channel) that has a shape to with the shape of the thermoplastic element is adapted so that the thermoplastic element during the process of being at least partially made flowable is guided by such guiding structure, and/or so that the flow of the flow portion of the thermoplastic material is guided.

In addition or as an alternative, the system may further include a guiding tool for such guiding. The guiding tool may have a defined position or a plurality of defined positions (to be assumed sequentially) relative to the support plate.

Especially, a guiding structure of the support plate and/or of a separate guiding tool may be such that the surgeon will place a plurality of the thermoplastic elements at defined positions around the periphery of the anchor implant.

In addition or as an alternative, the support plate and/or the anchor implant may include the thermoplastic material or a fraction thereof, for example as a thermoplastic collar of the anchor implant or as a thermoplastic collar of the support plate. It is also possible that the support plate and/or even the anchor implant is made of thermoplastic material. In many embodiments, however, the anchor implant includes a body of a not liquefiable, for example metallic material, such as Titanium or steel.

If the thermoplastic material, or a portion thereof, belongs to the support plate or possibly to the anchor implant, then it may still be accessible for an application tool (for example, a sonotrode) to directly impinge on the thermoplastic material, with the anchor implant anchored extending through the anchor receiving structure. Thereby, also in these embodiments both, the energy and a pressing force may be applied to the thermoplastic material to displace at least a portion thereof relative to the support plate body and the anchor implant.

More in general, at one of the following conditions may be fulfilled:
the thermoplastic material belongs to at least one thermoplastic element that is separate from the anchor implant and separate from the support plate, and the gap is accessible for the thermoplastic element to be pressed, by an application tool, against at least one of a surface portion of the support plate and of the anchor implant and for thermoplastic material thereof to be thereby caused to flow into the gap;
The support plate includes at least a portion of the thermoplastic material and further includes a support plate body of a not thermoplastic material, wherein the implant system further includes an application tool configured to impinge on the thermoplastic material for applying the energy and for exerting a pressing force on the thermoplastic material to move at least a portion thereof relative to the support plate body and the anchor implant.

Thus, the approach according to the present invention does not require a physical contact between the support plate and the anchor implant prior to the application of the energy but fulfills the above-cited requirement that it provides stability also in a situation where it is not possible to apply a pre-tension between the support plate and the anchor implant. Also, it does not require a forward movement of the anchor implant relative to the bone tissue and the support plate for the process. Rather, it makes possible that firstly the anchor implant is implanted, for example by screwing, extending through the support plate, and thereafter the securing by the thermoplastic material takes place.

To this end, the system may be configured for the thermoplastic material to be caused to flow relative to the anchor implant and the support plate, with the anchor implant and the support plate remaining stationary relative to one another, for the thermoplastic material to bridge and at least partially fil the gap.

Thus, the invention is especially suited for anchor implants that are bone screws with a screw head, the gap being a gap between the screw head and the support plate.

The gap bridged by the thermoplastic material may be a gap between lateral sides of the anchor implant and the anchor receiving structure and/or a gap between a distally facing shoulder of the anchor implant and the support plate. Such shoulder may especially be a shoulder formed by the head portion.

The gap may be, in the final state, bridged along a full circumference of the anchor implant, i.e., the thermoplastic material may form a continuous lining extending around the circumference of the anchor implant and around an inner contour of the anchor receiving structure. Alternatively, it may extend along a part of the periphery only, wherein preferably more than 180° of the circumference of the anchor implant is covered. As an even further alternative, the thermoplastic material may form a plurality of bridges around the contour.

In any case, the thermoplastic material may be distributed to take up forces in any lateral direction (direction parallel to the local bone surface plane) and optionally also relative axial forces between the anchor implant and the support plate in one direction (especially forces that pull the support plater relative to the anchor implant).

The energy to be applied to the thermoplastic material for making it flowable may be mechanical energy, especially mechanical vibration energy. For applying the energy, a vibrating tool (sonotrode) press the thermoplastic material into the gap.

Other forms of energy, such as radiation energy, inductive heat, etc., are not excluded.

The system may further include an energy application tool, for example sonotrode, for applying the energy, wherein the energy application tool is configured to impinge on the thermoplastic material for applying the energy and for exerting a pressing force on the thermoplastic material to move at least a portion thereof relative to the support plate and the anchor implant.

Such energy application tool may include a distally facing outcoupling surface with a shape adapted to the thermoplastic material. For example, if the thermoplastic material is a separate element, the application tool may be adapted to such separate element. If the thermoplastic material forms a collar around the anchor implant (for example a collar belonging to the support plate), then the application tool may include a concave outcoupling shape following the shape of the collar.

The support plate will in embodiments be a flattish element adapted to the surface of the vertebral bone and lying flattishly, in surroundings of the anchor implant, against the vertebral bone by following its shape. Compared to a washer, the support plate may be larger, with an area along the bone surface contour for example being at least 3 times or at least 5 or even at least 8 times a cross section area of the anchor implant in section along the bone surface plane.

Especially, the support plate may be shaped or shapeable (for example by thermic or plastic deformation) to have a 3D surface with a curvature following the bone surface. An alternative manufacturing technique is additive manufacturing. Additive manufacturing may be interesting especially in cases of complex requirements, which may depend on the patient's anatomy (see hereinafter). Also, if the geometry of the support plate itself is more complex, for example by including undercut structures, through holes, spikes, thickness variations etc., additive manufacturing may be a good alternative to deformation.

The support plate may be adapted to run along a surface portion of one single vertebra, i.e. not to form any bridge between different vertebrae. If the implant system includes a plurality of the fasteners and an according plurality of fastening locations, all fasteners are anchored in the same vertebra. By this, the support plate does not restrict any degree of freedom in the correction of the relative positions of the vertebrae of the spinal column.

In embodiments, the support plate is custom manufactured to be adapted to the size and shape of the patient's vertebra.

To this end a method of obtaining an implant system according to any embodiment of the invention may include choosing, relative to the patient's spinal column, an implantation location for implanting the anchor implant, of obtaining information on bone size and shape of the patient, of choosing an adapted support plate shape and size and, and using taking the support plate of the adapted support plate shape.

In this, obtaining the information may include using a 3D-imaging process for obtaining 3D image data on the patient and/or taking the support plate of the adapted s shape and size may include custom manufacturing the support plate.

As an alternative, the data, especially for standard cases, may be obtained based on well-known information on average sizes and properties.

In embodiments, the implant system further includes at least one fastener in addition to the anchor implant. Such fastener may especially be a fastener of the kind that includes a thermoplastic material in a solid state and is equipped for being anchored in bone tissue of a patient in an anchoring process that includes applying a pressing force and coupling energy, such as mechanical vibration energy, into the fastener. The thermoplastic material of the fastener may be the same as the thermoplastic material that is used to bridge the gap between the anchor implant and the support plate, or it may be different therefrom.

In embodiments, such a fastener includes the thermoplastic material at least at the distal end thereof and is anchored by being pressed against bone tissue by a pressing force acting from a proximal side, and by energy coupled into the fastener to at least partially liquefy the fastener thermoplastic material, wherein a flow portion of the fastener thermoplastic material is pressed into bone tissue and, after re-solidification, anchors the fastener in the bone tissue, and wherein the fastener is equipped for cooperating with the fastening structure to secure the support plate to the posterior or lateral side of the spinal column. In addition or as an alternative, the fastener may include a body of a non-liquefiable material with an opening accessible from the proximal side and with channels from the opening to an outer surface, whereby the thermoplastic material is pressable from the opening out of the channels into the tissue to anchor the fastener.

In embodiments, a fastener of the described kind may be longer than a depth of the bone tissue. A length of the fasteners thus may be sufficient for the distal end face to reach through an opening in a proximal cortical bone portion of the bone tissue and through cancellous bone of the bone tissue to be pressed against a distal cortical bone portion of the bone tissue. This may especially pertain to the position of the fastener in the bone tissue of the patient's lamina, vertebral processes, ribs or occiput, as defined by the respective fastening structure.

The energy used for the anchoring process of a fastener of the mentioned kind may be mechanical vibration energy. To this end, the fastener may include a proximally facing coupling-in face.

The fastener may be anchored prior to positioning the support plate relative to the tissue. Then, the implantation method includes the additional step of securing the support plate to the fasteners. The fastening structures may then be undercut structures that optionally may be restricted to distal side. This latter option makes possible that the proximal surface is smooth also at the locations of the anchoring structures, so that irritation of soft tissue is minimized.

Alternatively, the fasteners may be anchored after positioning the support plate, for example through through openings in the support plate, which through openings constitute the fastening structures. Such through openings as fastening structures may possibly be broadened towards the proximal side so that a head of the respective fastener may be countersunk.

In a group of embodiments, the fasteners may include an opening extending inwardly, towards proximally, from the distal end. Thus, the fasteners may have a split or cannulated distal end.

The invention also concerns a method of implanting an anchor implant in a human or animal vertebra and of stabilizing the anchor implant relative to the bone tissue of the vertebra by means of a support plate. The method includes the steps of placing the support plate relative to the bone tissue, anchoring the anchor implant in the bone tissue (in either sequence), so that a gap remains between the support plate and the anchor implant, and causing energy to impinge on thermoplastic material until a flow portion thereof becomes flowable and flows into the gap and/or in the gap until the gap is bridged by the thermoplastic material, of stopping the energy transfer and causing the thermoplastic material to re-solidify, whereby the anchor implant is supported by the support plate via the re-solidified thermoplastic material bridging the gap.

In this, the energy may be mechanical vibration energy, applied by a vibrating tool.

The method may in addition include pressing the thermoplastic material towards the gap and into the gap. To this end, if applicable, the vibrating tool may press the thermoplastic material into the gap.

Mechanical vibration or oscillation suitable for devices according to embodiments of the invention and according methods that include liquefaction of a polymer by friction heat created through the mechanical vibration has preferably a frequency between 2 and 200 kHz (even more preferably between 10 and 100 kHz, or between 20 and 40 kHz) and a vibration energy of 0.2 to 20 W per square millimeter of active surface. The vibrating element (sonotrode) is, e.g., designed such that its contact face oscillates predominantly in the direction of the element axis (longitudinal vibration) and with an amplitude of between 1 and 100 μm, preferably around 10 to 30 μm. Rotational or radial oscillation is possible also.

For specific embodiments of devices, it is possible also to use, instead of mechanical vibration, a rotational movement for creating the named friction heat needed for the liquefaction of the anchoring material. Such rotational movement has preferably a speed in the range of 10,000 to 100,000 rpm.

A further way for producing the thermal energy for the desired liquefaction includes coupling electromagnetic radiation into one of the device parts to be implanted and designing one of the device parts to be capable of absorbing the electromagnetic radiation, wherein such absorption preferably takes place within the anchoring material to be liquefied or in the immediate vicinity thereof. Preferably electromagnetic radiation in the visible or infrared frequency range is used, wherein the preferred radiation source is a corresponding laser. Electric heating of one of the device parts may also be possible.

In this text the expression “thermoplastic material being liquefiable, e.g., by mechanical vibration” or in short “liquefiable thermoplastic material” or “liquefiable material” is used for describing a material including at least one thermoplastic component, which material becomes liquid (flowable) when heated, in particular when heated through friction, i.e., when arranged at one of a pair of surfaces (contact faces) being in contact with each other and vibrationally or rotationally moved relative to each other, wherein the frequency of the vibration is between 2 kHz and 200 kHz, preferably 20 to 40 kHz and the amplitude between 1 μm and 100 μm, preferably around 10 to 30 μm. Such vibrations are, e.g., produced by ultrasonic devices as is, e.g., known for dental applications.

In this text, generally a “non-liquefiable” material is a material that does not liquefy at temperatures reached during the process, thus especially at temperatures at which the thermoplastic material of the fastener is liquefied. This does not exclude the possibility that the non-liquefiable material would be capable of liquefying at temperatures that are not reached during the process, generally far (for example by at least 80° C.) above a liquefaction temperature of the thermoplastic material or thermoplastic materials liquefied during the process. The liquefaction temperature is the melting temperature for crystalline polymers. For amorphous thermoplastics the liquefaction temperature is a temperature above the glass transition temperature at which the becomes sufficiently flowable, sometimes referred to as the ‘flow temperature’ (sometimes defined as the lowest temperature at which extrusion is possible), for example the temperature at which the viscosity drops to below 10⁴ Pa*s (in embodiments, especially with polymers substantially without fiber reinforcement, to below 10³ Pa*s)), of the thermoplastic material.

For example, a non-liquefiable material may be a metal, or ceramic, or a hard plastic, for example a reinforced or not reinforced thermosetting polymer or a reinforced or not reinforced thermoplastic with liquefaction temperature considerably higher than the liquefaction temperature of the liquefiable material, for example with a melting temperature and/or glass transition temperature higher by at least 50° C. or 80° C. or 100° C.

For being able to constitute a load-bearing connection to the tissue, the material has an elasticity coefficient of more than 0.5 GPa, preferably more than 1 GPa. The elasticity coefficient of at least 0.5 GPa also ensures that the liquefiable material is capable of transmitting the ultrasonic oscillation with such little damping that inner liquefaction and thus destabilization of the liquefiable element does not occur, i.e. liquefaction occurs only where the liquefiable material is at the liquefaction interface to the stop face. The plastification temperature is preferably of up to 200° C., between 200° C. and 300° C. or even more than 300° C. Depending on the application, the liquefiable thermoplastic material may or may not be resorbable.

Suitable resorbable polymers are e.g. based on lactic acid and/or glycolic acid (PLA, PLLA, PGA, PLGA etc.) or polyhydroxyalkanoates (PHA), polycaprolactones (PCL), polysaccharides, polydioxanones (PD), polyanhydrides, polypeptides or corresponding copolymers or blended polymers or composite materials containing the mentioned polymers as components are suitable as resorbable liquefiable materials. Thermoplastics such as for example polyolefins, polyacrylates, polymetacrylates, polycarbonates, polyamides, polyesters, polyurethanes, polysulphones, polyaryl ketones, polyimides, polyphenyl sulphides or liquid crystal polymers (LCPS), polyacetals, halogenated polymers, in particular halogenated polyoelefins, polyphenylene sulphides, polysulphones, polyethers, polypropylene (PP), or corresponding copolymers or blended polymers or composite materials containing the mentioned polymers as components are suitable as non-resorbable polymers. Examples of suited thermoplastic material include any one of the polylactide products LR708 (amorphous Poly-L-DL lactide 70/30), L209 or L210S by Bohringer Ingelheim.

Specific embodiments of degradable materials are Polylactides like LR706 PLDLLA 70/30, R208 PLDLA 50/50, L210S, and PLLA 100% L, all of Bohringer. A list of suitable degradable polymer materials can also be found in: Erich Wintermantel und Suk-Woo Haa, “Medizinaltechnik mit biokompatiblen Materialien und Verfahren”, 3. Auflage, Springer, Berlin 2002 (in the following referred to as “Wintermantel”), page 200; for information on PGA and PLA see pages 202 ff., on PCL see page 207, on PHB/PHV copolymers page 206; on polydioxanone PDS page 209. Discussion of a further bioresorbable material can for example be found in CA Bailey et al., J Hand Surg [Br] 2006 April; 31(2):208-12.

Specific embodiments of non-degradable materials are: Polyetherketone (PEEK Optima, Grades 450 and 150, Invibio Ltd), Polyetherimide, Polyamide 12, Polyamide 11, Polyamide 6, Polyamide 66, Polycarbonate, Polymethylmethacrylate, Polyoxymethylene, or polycarbonateurethane (in particular Bionate® by DSM, especially Bionate 75D and Bionate 65D; according information is available on datasheets publicly accessible for example via www.matweb.com by Automation Creations, Inc.). An overview table of polymers and applications is listed in Wintermantel, page 150; specific examples can be found in Wintermantel page 161 ff. (PE, Hostalen Gur 812, Hochst AG), pages 164 ff. (PET) 169 ff. (PA, namely PA 6 and PA 66), 171 ff. (PTFE), 173 ff. (PMMA), 180 (PUR, see table), 186 ff. (PEEK), 189 ff. (PSU), 191 ff. (POM—Polyacetal, tradenames Delrin, Tenac, has also been used in endoprostheses by Protec).

The liquefiable material having thermoplastic properties may contain foreign phases or compounds serving further functions. In particular, the thermoplastic material may be strengthened by admixed fillers, for example particulate fillers that may have a therapeutic or other desired effect. The thermoplastic material may also contain components which expand or dissolve (create pores) in situ (e.g., polyesters, polysaccharides, hydrogels, sodium phosphates) or compounds to be released in situ and having a therapeutic effect, e.g. promotion of healing and regeneration (e.g., growth factors, antibiotics, inflammation inhibitors or buffers such as sodium phosphate or calcium carbonate against adverse effects of acidic decomposition). If the thermoplastic material is resorbable, release of such compounds is delayed.

If the liquefiable material is to be liquefied not with the aid of vibrational energy but with the aid of electromagnetic radiation, it may locally contain compounds (particulate or molecular), which are capable of absorbing such radiation of a specific frequency range (in particular of the visible or infrared frequency range), e.g., calcium phosphates, calcium carbonates, sodium phosphates, titanium oxide, mica, saturated fatty acids, polysaccharides, glucose or mixtures thereof.

Fillers used may include degradable, osseostimulative fillers to be used in degradable polymers, including: β-Tricalciumphosphate (TCP), Hydroxyapatite (HA, <90% crystallinity; or mixtures of TCP, HA, DHCP, Bioglasses (see Wintermantel). Osseo-integration stimulating fillers that are only partially or hardly degradable, for non degradable polymers include: Bioglasses, Hydroxyapatite (>90% cristallinity), HAPEX®, see SM Rea et al., J Mater Sci Mater Med. 2004 September; 15(9):997-1005; for hydroxyapatite see also L. Fang et al., Biomaterials 2006 July; 27(20):3701-7, M. Huang et al., J Mater Sci Mater Med 2003 July; 14(7):655-60, and W. Bonfield and E. Tanner, Materials World 1997 January; 5 no. 1:18-20. Embodiments of bioactive fillers and their discussion can for example be found in X. Huang and X. Miao, J Biomater App. 2007 April; 21(4):351-74), JA Juhasz et al. Biomaterials, 2004 March; 25(6):949-55. Particulate filler types include: coarse type: 5-20 μm (contents, preferentially 10-25% by volume), sub-micron (nanofillers as from precipitation, preferentially plate like aspect ratio >10, 10-50 nm, contents 0.5 to 5% by volume).

A specific example of a material with which experiments were performed was PLDLA 70/30 including 30% (weight percent) biphase Ca phosphate that showed a particularly advantageous liquefaction behaviour.

The material of the anchor implant and the material of the support plate may be any material being suitable for surgical applications and being sufficiently stiff. For example, the anchor implant and the support plate may be of any material that does not melt at the melting temperatures of the thermoplastic material. The anchor implant and the support plate may be of equal or different materials/material compositions.

Especially, the anchor implant and/or the support plate may be of a metal, for example a titanium alloy. A preferred material is titanium grade5. Alternative materials are other metals like other titanium alloys, stainless steel, or hard plastics such as PEEK etc.

### BRIEF DESCRIPTION OF THE DRAWINGS

In the following, ways to carry out the invention and embodiments are described referring to drawings. The drawings mostly are schematic. In the drawings, same reference numerals refer to same or analogous elements. The drawings show:

FIG. 1 in schematical horizontal section, a human vertebra with a pedicle screw and a support plate;

FIGS. 2 and 3 in schematical section, close-ups of arrangements of a pedicle screw and a support plate during the set of causing the anchor implant to be supported;

FIGS. 4 and 5 details of a further arrangement;

FIG. 6 a sonotrode of an arrangement as shown in FIG. 5;

FIG. 7 a further implanted implant system with a pedicle screw and a support plate;

FIG. 8 schematically, an even further configuration;

FIGS. 9 and 10 yet another configuration; and

FIG. 11 a configuration with a guiding tool in addition to the support plate.

### DESCRIPTION OF THE PREFERRED EMBODIMENTS

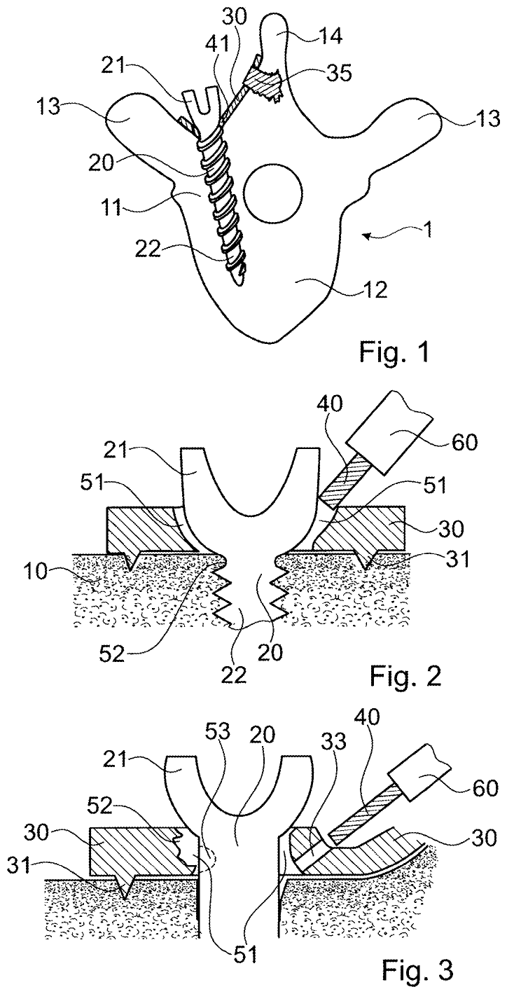

FIG. 1 shows a human vertebra 1 with an anchor implant, namely a pedicle screw 20 (as an alternative to a pedicle screw, the anchor implant may have another structure, not necessarily having a thread, see for example FIGS. 13-17 of WO 2011/054124). The pedicle screw extends from a posterior side of the vertebra through a pedicle 11 into the vertebral body 12. The pedicle screw includes a proximal head portion 21 equipped for cooperating with a spine stabilizer, such as a rod. From the head portion 21, a threaded shaft portion 22 extends towards distally into the bone tissue.

For supporting the pedicle screw, a support plate 30 is implanted on the posterior side of the vertebral body. The support plate 30 is anchored in and/or supported by bone tissue of the vertebra, especially one of the transverse processes 13 and/or the spinous process 14.

For anchoring the support plate—in addition to the anchoring caused by the anchor implant —, at least one thermoplastic fastener 35 extends through an attachment structure being a fastener through opening in the depicted embodiment.

A thermoplastic adjustment element 41 is in the gap between the head portion and possibly also the proximal-most section of the anchor implant on the one hand and the support plate 30 on the other hand. The thermoplastic adjustment element may for example be circumferential, i.e. extend 360° around the anchor implant. Also, it completely bridges the gap between the anchor implant and the support plate so that a least for some directions forces that act on the head portion of the anchor implant are partially coupled into the support plate 30. Thereby, the anchor implant is supported by a much larger section of the—comparably hard—cortical bone of the posterior and/or lateral surface of the vertebra than if the head portion would directly be pressed against the bone tissue or the head portion would be supported by a washer only or if the head portion would not be supported by the tissue at all (but just the shaft portion would be supported).

Also, due to the adjustment element 41 there is an additional degree of freedom because the support plate may support the anchor implant even if the size of the gap is not precisely known in advance, for example because the head portion includes an uniaxial head the orientation of which is pre-defined by the function it has to carry out.

FIG. 2 schematically illustrates a method of causing the anchor implant to be supported by the support plate 30 using an adjustment element. The support plate 30 includes a through opening. For implantation, firstly the support plate is placed relative to the bone tissue 10 of the vertebra. Then, the elongate anchor implant, here again illustrated to be a pedicle screw 20, is advanced through the through opening into the tissue until it has reached the desired position and is implanted in the bone tissue. A gap 51 may remain between the head portion 21 and/or the shaft portion 22 of the anchor implant on the one hand and the support plate 30 on the other hand.

Thereafter, an initially separate thermoplastic element 40—here illustrated to be a thermoplastic pin—is placed relative to the support plate 30 and the anchor implant and then is subject to energy input while being pressed into the gap 51 and against the anchor implant or the support plate or both. As a consequence, material of the thermoplastic element 40 becomes flowable and flows into the gap 51 and/or in the gap 51 until it bridges the gap, for example around a full periphery of the anchor implant. After the energy input stops, the thermoplastic material re-solidifies and forms the adjustment element, and the anchor implant is supported by the support plate via the thermoplastic material.

In FIG. 2 as well as in other illustrations referred to in this text, the input energy is assumed to be mechanical vibration energy. To this end, a vibrating tool 60 (sonotrode) is schematically sketched. The vibrating tool 60 is used to press the thermoplastic element towards the gap while mechanical vibration is coupled into it.

The embodiment of FIG. 2 has a further feature that is an option for any embodiment. Namely, the support plate 30 has at least one spike 31 penetrating into the bone tissue. Such spike or other penetrating feature has the effect of stabilizing the support plate with respect to shear forces.

In FIG. 2, the thermoplastic element is depicted to be pressed directly into the gap 51 from the proximal side thereof. In the embodiment of FIG. 3, in contrast, the support plate 30 has a through channel 33 connecting a proximal end face of the support plate with the gap 51. Then, the thermoplastic element 40 may be pressed into the gap through this through channel 33.

A further, independent optional feature of embodiments of the invention is also illustrated in FIG. 3. Namely, the support plate 30 forms an undercut 52 with respect to axial directions around at least a part of the periphery of the attachment structure. Thereby, after the gap is filled by thermoplastic material, an additional stabilization with respect to axial directions is achieved. In addition or as an alternative, the anchor implant 20 may include an undercut 53, whereby axial loads on the anchor implant (towards proximally) may be absorbed after the undercut is filled by thermoplastic material.

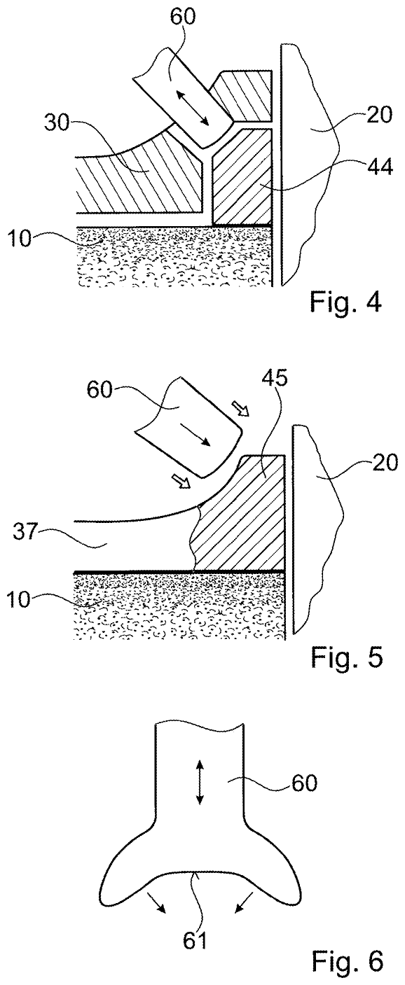

FIG. 4 depicts a variant in which the thermoplastic material is present not as a pin-like thermoplastic element but as a thermoplastic ring 44 extending around the anchor implant 20. In the depicted embodiment, the thermoplastic element is restricted to the distal side of the support plate 30 so that the support plate 30 forms a collar around the anchor implant proximally of the thermoplastic ring 44. For the energy input, the thermoplastic ring 44 is accessed, for example by a vibrating tool 60, through at least one through hole in the support plate.

In the embodiment of FIG. 5, the thermoplastic material is not a separate element but belongs to the support plate. It forms a thermoplastic collar 45 around the anchor implant. In addition to the thermoplastic collar, the support plate may include portions (a support plate body) of not thermoplastic material 37 to which the thermoplastic collar 45 is secured. Alternatively, the support plate may be entirely thermoplastic.

The thermoplastic collar 45 in FIG. 5 forms an optional proximal protrusion.

For impinging a thermoplastic collar 45—or an initially separate annular thermoplastic element or other thermoplastic structure that at least partially encompasses the anchor implant ab initio—with mechanical vibration energy, a vibrating tool 60 of the kind illustrated in FIG. 6 may be used. FIG. 6 shows the vibrating tool 60 of FIG. 5 with a curved outcoupling face. The radius of the interior outcoupling face 61 may be approximately equal to or slightly smaller than an outer radius of the collar 45. For the process, the vibrating tool 60 is pressed against the thermoplastic collar or other at least partially annular structure in the direction of the arrows in FIG. 6.

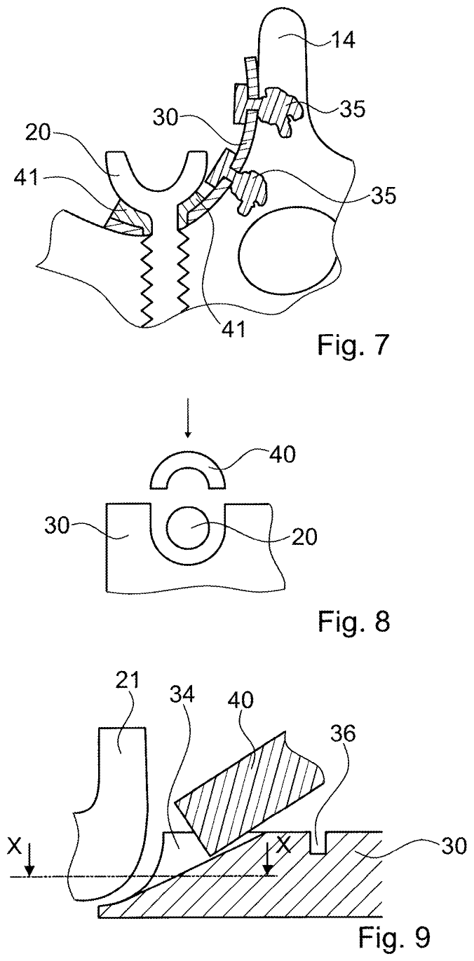

FIG. 7 illustrates in somewhat more detail a configuration of an anchor implant 20 being a pedicle screw with a support plate that extends along the spinous process 14. The thermoplastic adjustment element 41 fills the gap, both, between the proximal-most part of the shaft portion and the through opening in the support plate as well as between the distally facing shoulder formed by the head portion on the one hand and the support plate 30 on the other hand.

The support plate is in addition fastened to the bone tissue by at least one fastener 35—two fasteners 35 are illustrated in FIG. 7. The fasteners 35 are anchored in the bone tissue by being positioned through corresponding fastening holes of the support plate 30 and then being pressed into the bone tissue while energy is coupled into them. Prior to the step of bringing the fastener into contact with the bone tissue, the proximally-facing portion of the cortical bone may be locally removed to expose the cancellous bone tissue underneath. This may be done by any, for example conventional, surgical means. In an alternative to conventional means, it may be done assisted by ultrasound.

Thereafter, the fastener is anchored with respect to the bone tissue. In accordance with a first possibility, this may be done after the support plate has been placed. Optionally, already the removal of cortical bone tissue may be done with the support plate positioned relative to the bone tissue. The support plate may then serve as a kind of template. In accordance with a second possibility, the support plate may be poisoned after anchoring. Then, instead of the fastening holes being through openings, the support plate includes other fastening structures, for example distally facing undercut structures.

For the anchoring process, energy is coupled into the fastener while the fastener is pressed towards distally. This may for example be done by a vibrating tool. The step of coupling energy into the fastener is continued or repeated until the thermoplastic material is sufficiently heated for a flow portion thereof to become flowable. Due to the pressing force, the thermoplastic material is displaced. In this, the cortical bone tissue distally of the location from where the bone tissue is accessed may serve as an abutment and as a stop, as for example described in Swiss patent application 01159/16.

After the energy input stops, the thermoplastic material re-solidifies and anchors the respective fastener in the bone tissue, by the re-solidified thermoplastic material interpenetrating structures of the cancellous bone tissue, and/or by the thermoplastic material forming a broadening distally of the proximally-facing portion of the cortical bone, whereby a blind rivet effect is achieved.

The fastener 35 has a proximal structure cooperating with the support plate 30 to secure the support plate against the bone tissue, for example a proximal head as illustrated. Such structure may be pre-manufactured or formed, for example by the vibrating tool, in situ.

FIG. 8 illustrates the principle that the anchor receiving structure of the support plate 30 does not need to be a hole completely surrounded by a rim but can, for example, also be a recess that is open towards one side. Preferably, the rim extends around such a recess by more than 180°. A separate thermoplastic element 40 may be adapted thereto in shape, as schematically sketched in FIG. 8.

The illustrated embodiments have illustrated the anchor implant to be a pedicle screw with a uniaxial head, i.e., a head solidly connected to the shaft portion. However, the invention also works for other anchor implants, including pedicle screws with multiaxial head, other spinal bone screws, anchor implants of the kind described in WO 2011/054124, etc.

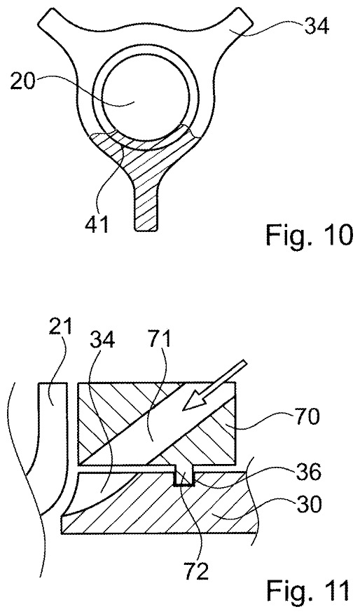

The embodiment of FIGS. 9 and 10 (FIG. 10 illustrating a cross section through plane X-X in FIG. 9), distinguishes from the embodiment of FIG. 2 in that the support plate 30 has a plurality of guide channels 34. The guide channels are adapted to the thermoplastic elements 40 so that the thermoplastic elements are guided. Also, they define the position of the thermoplastic elements with respect to the support plate around the pedicle screw 20. For example, as shown in FIG. 10, the channels may be distributed at equal distances around the periphery so that the adjustment element 41 portions formed by the different thermoplastic elements mechanically support the pedicle screw with respect to the support plate in a well-balanced manner.

For additional guidance, a separate guiding tool 70 may be used in addition or as an alternative to the guide channels 34 (or the through channels in embodiments like the one of FIG. 3.) Such guiding tool will be place relative to the support plate in a defined position. To this end, a positioning structure may be present. In FIG. 11, the support plate 30 has a positioning hole 36 cooperating with a positioning protrusion 72 of the guiding tool to define the position of the guiding tool. The guiding tool includes a guiding hole 71 that may, if applicable be aligned with the respective guide channel (or through channel). Thereby, the location and orientation of the thermoplastic element 40 during the process are defined, and the material flow is controlled, especially if such guidance/flow control cannot be sufficiently achieved by the structure of the support plate alone.

In FIG. 1 and in the description of embodiments of the invention, the anchor implant 20 is assumed to be a pedicle screw that extends from the posterior side into the vertebral body. This is not a requirement. The invention also pertains to other anchor implants that do not necessarily extend into the vertebral body, for example shorter screws, lateral screws, anchor implants that are not screws (for example, according to the principle shown in FIGS. 13-17 of WO 2011/054124), etc.

1. A spinal implant system, comprising:
a spinal anchor implant extending between a proximal end and a distal end and shaped to be anchored in a human spine with the distal end extending into the bone tissue of a vertebra from a posterior or lateral side thereof, the spinal anchor implant having a proximal portion forming the proximal end;
and a support plate, the support plate being shaped to be placed against the posterior or lateral side of the vertebra, the support plate having an anchor receiving structure, wherein the anchor implant extends through the anchor receiving structure into the bone tissue when the anchor implant is anchored in the bone tissue;
wherein the implant system comprises a thermoplastic material in a solid state, the thermoplastic material being shaped and equipped to be placed relative to the anchored anchor implant and the support plate and for at least a flow portion of which to be made flowable by the impact of impinging energy so as to bridge and at least partially fill a gap between the anchor implant and the support plate;
whereby after re-solidification of the thermoplastic material, the support plate supports the anchored anchor implant in a force transmitting manner via the thermoplastic material.

2. The implant system according to claim 1, wherein the anchor implant is elongate with a longitudinal axis extending proximodistally and may be a pedical screw.

3. (canceled)

4. The implant system according to claim 1, wherein the thermoplastic material belongs to at least one thermoplastic element that is separate from the anchor implant and separate from the support plate.

5. The implant system according to claim 1, wherein the at least one of the anchor implant and of the support plate comprises the thermoplastic material.

6. The implant system according to claim 1, wherein at least one of the support plate and the anchor implant forms an undercut along at least a part of the gap.

7. (canceled)

8. The implant system according to claim 6, wherein the undercut comprises an undercut with respect to axial directions.

9. The implant system according to claim 1, wherein the gap is a gap between lateral sides of the anchor implant on the one hand and the anchor receiving structure on the other hand, and/or is a gap between a distally facing shoulder of the anchor implant on the one hand and the support plate on the other hand.

10. The implant system according to claim 1, wherein after re-solidification the thermoplastic material bridges the gap around a full circumference of the anchor implant.

11-12. (canceled)

13. The implant system according to claim 1, wherein an area of the support plate is at least 5 times a cross section area of the anchor implant.

14. The implant system according to claim 1, further comprising at least one fastener equipped for being anchored in the bone tissue, wherein the support plate comprises at least one fastening location, and wherein the fastener is equipped for cooperating with the fastening location to secure the anchoring plate against the bone tissue when the fastener is anchored in the bone tissue.

15. The implant system according to claim 14, wherein the fastener comprises a fastener thermoplastic material in a solid state and is equipped for being anchored in bone tissue in an anchoring process by being pressed against bone tissue by a pressing force acting from a proximal side, and by energy coupled into the fastener to at least partially liquefy the fastener thermoplastic material, wherein a flow portion of the fastener thermoplastic material is pressed into bone tissue and, after re-solidification, anchors the fastener in the bone tissue, and wherein the fastener is equipped for cooperating with the fastening structure to secure the support plate to the posterior or lateral side of the spinal column.

16. The implant system according to claim 14, comprising a plurality of the fasteners.

17. (canceled)

18. The implant system according to claim 1, wherein when the anchor implant extends through the anchor receiving structure into the bone tissue when the anchor implant is anchored in the bone tissue and when the support plate is placed against the posterior or lateral side of the vertebra, the support plate extends into a region between the proximal portion of the spinal anchor implant being a head portion and the bone tissue.

19. The implant system according to claim 1, wherein the thermoplastic material belongs to at least one thermoplastic element that is separate from the support plate, and wherein the support plate has a guiding structure defining a location and orientation of the thermoplastic element when flow portion of the thermoplastic material is made flowable or the thermoplastic material belongs to at least one thermoplastic element that is separate from the support plate, wherein the implant system further comprises a guiding tool with a guiding structure, defining a location and orientation of the thermoplastic element when flow portion of the thermoplastic material is made flowable.

20. (canceled)

21. The implant system according to claim 19, wherein the guiding tool has a positioning structure cooperating with a support plate positioning structure of the support plate to define a position of the guiding tool relative to the support pate.

22. The implant system according to claim 1, wherein the anchor implant is a bone screw configured to be anchored in the bone tissue extending through the anchor receiving structure, the anchor implant having a screw head securing the support plate against the bone tissue when the anchor implant is anchored in the bone tissue extending through the anchor receiving structure.

23. The implant system according to claim 1, being configured for the thermoplastic material to be caused to flow relative to the anchor implant and the support plate, with the anchor implant and the support plate remaining stationary relative to one another, for the thermoplastic material to bridge and at least partially fill the gap.

24. The implant system according to claim 1, wherein the support plate is one-piece at least around the anchoring location or at least comprises a one-piece support plate body extending around the anchoring location.

25. The implant system according to claim 1, and further comprising an energy application tool for applying the energy, wherein the energy application tool is configured to impinge on the thermoplastic material for applying the energy and for exerting a pressing force on the thermoplastic material to move at least a portion thereof relative to the support plate and the anchor implant.

26. (canceled)

27. The implant system according to claim 1, wherein at least one of the following conditions is fulfilled:
the thermoplastic material belongs to at least one thermoplastic element that is separate from the anchor implant and separate from the support plate, and the gap is accessible for the thermoplastic element to be pressed, by an application tool, against at least one of a surface portion of the support plate and of the anchor implant and for thermoplastic material thereof to be thereby caused to flow into the gap;
the support plate comprises at least a portion of the thermoplastic material and further comprises a support plate body of a not thermoplastic material, wherein the implant system further comprises an application tool configured to impinge on the thermoplastic material for applying the energy and for exerting a pressing force on the thermoplastic material to move at least a portion thereof relative to the support plate body and the anchor implant.

28. A method of obtaining an implant system according to claim 1, the method comprising choosing an implantation location relative to a patient's spinal column, of obtaining information on bone size and shape of the patient, of choosing an adapted support plate shape and size and an anchor implant size, and of taking the support plate of the adapted support plate shape and size and the anchor implant of the adapted anchor implant size.

29. (canceled)

30. The method according to claim 28, wherein taking the support plate of the adapted support plate shape and size comprises custom manufacturing the support plate.

31. The method according to claim 30, wherein custom manufacturing comprises using 3D-data to shape the implant body adapted to the patient's skeleton or comprises choosing a pre-defined implant body shape, for example from a plurality of pre-defined implant body shapes, and of adding fastening structures.

32. (canceled)

33. A method of implanting an anchor implant in a human or animal vertebra and of stabilizing the anchor implant relative to the bone tissue of the vertebra via a support plate, the method comprising the steps of:
placing the support plate relative to the bone tissue and anchoring the anchor implant in the bone tissue, so that a gap remains between the support plate and the anchor implant,
causing energy to impinge on thermoplastic material until a flow portion thereof becomes flowable and flows into the gap and/or flows in the gap until the gap is bridged by the thermoplastic material,
stopping the energy transfer, and
causing the thermoplastic material to re-solidify, whereby the anchor implant is supported by the support plate via the re-solidified thermoplastic material bridging the gap.

34. (canceled)

35. The method according to claim 33, further comprising fastening the support plate to vertebral bone tissue by a fastener.

36. The method according to claim 35, wherein the fastener comprises a fastener thermoplastic material in a solid state, and wherein the method further comprises anchoring the fastener in bone tissue by pressing the fastener against bone tissue by a pressing force acting from a proximal side, and coupling energy into the fastener to at least partially liquefy the fastener thermoplastic material, until a flow portion of the fastener thermoplastic material is pressed into bone tissue and, after re-solidification, anchors the fastener in the bone tissue, and wherein the method further comprises causing the fastener to cooperate with the fastening structure to secure the support plate to the posterior or lateral side of the spinal column.
